(19) United States
(12) Patent Application Publication
(10) Pub. No.: US 2022/0061722 A1
(43) Pub. Date: Mar. 3, 2022

(54) BLOODLETTING SYRINGE, BLOODLETTING SYRINGE SYSTEM AND BLOODLETTING METHODS

(71) Applicant: Wellness Allied Inc, Natick, MA (US)

(72) Inventors: Weihui Li, Natick, MA (US); Tao ZHANG, Natick, MA (US)

(21) Appl. No.: 17/006,970

(22) Filed: Aug. 31, 2020

(51) Int. Cl. A61B 5/153; A61B 5/15

(52) U.S. Cl. CPC A61B 5/153; A61B 5/150229; A61B 5/150358; A61B 5/150511; A61B 5/150412; A61B 5/150381; A61B 5/150389

(57) ABSTRACT

A bloodletting syringe, including: a hollow needle that can be placed under skins, a barrel connected to the hollow needle, and a pump outlet being connected to the barrel, wherein the pump outlet is a one-way port. When pumping, the pump outlet is opened, air is pumped out of the barrel and hollow needle, when the pumping is stopped, the pump outlet is automatically closed; air pressure in the barrel becomes lower than the pressure outside of the barrel. The bloodletting syringe, bloodletting syringe systems and bloodletting methods of the present invention can be used to remove blood stasis in muscles and tissues safely and with great control by medical staff.

### TECHNICAL FIELD

The present invention generally relates to a bloodletting syringe, a bloodletting syringe system and a bloodletting method to treat blood stasis related diseases.

### BACKGROUND

Blood-letting therapy has a long history. As early as more than two thousand years ago, there is a record of the bloodletting in the book “The Emperor's Inner Classic” from China. In English language world, the famous medical journal, Lancet, is named after the bloodletting therapy tool, lancet. Bloodletting therapy improves local blood circulation by removing blood stasis from a local tissue, allowing oxygenated blood to flow into the area. Bloodletting is usually performed with a triangular needle or lancet to pierce the skin and let out the blood from the capillaries/venules or interstitial tissue. FIG. 1A shows the shape of a lancet and FIG. 1B shows the shape of a triangular needle. The lancet and the triangular needle are solid with length being around a few millimeters. Therefore, these types of bloodletting tools are generally used to remove blood stasis in superficial tissue. But some tissues in the body can be as thick as a few centimeters to a dozen centimeters, such as biceps, triceps, or gluteus maximus. With the lancet or triangular needle, it is hard to clear out blood stasis efficiently from these big muscles or deep tissues.

Another bloodletting method is commonly called bloodletting cupping. It performs cupping with a cupping cup after piercing the skin with a lancet or a triangular needle. The air inside of the cupping cup is pumped out to create a lower air pressure, which will lift the tissue underneath the cup and squeeze out the blood from deeper tissues. FIG. 2A is a diagram of bloodletting cupping, 210 is a cupping cup and 220 are some drops of blood pulled out from the tissue. FIG. 2B is a manual air pumper.

In modern medicine, syringes are usually used to inject drugs in a liquid form into the human body. FIG. 3 shows a common syringe with a hollow needle. 310 is a tip of the needle, 320 is a shaft of the needle, which is usually about a few centimeter long. Connected with the needle is a needle hub 325 and then a barrel 330. A plunger seal 340 can be moved within the barrel 330 when pulling or pushing a plunger 350. As the name “plunger seal” indicates, the barrel 330 is sealed with the plunger seal and no air or liquid can be leaked through the plunger seal. The syringe needle is sharp to enable it to puncture through the skin and blood vessels easily. Another application of syringe is to draw blood from vessels. When in use, a medical staff pulls the plunger out to increase the space in the barrel and decrease the internal air pressure. It is the lower internal air pressure within the barrel that draws the blood out from the body. During the drawing of the blood, the medical staff needs to hold the plunger all the time. Otherwise, the lower air pressure inside the barrel will pull the plunger back in to the barrel and stop the process of blood drawing.

### SUMMARY

In view of the triangular needle, lancet used for bloodletting, the bloodletting cupping and the syringe for injection and blood drawing, the present invention proposes a bloodletting syringe, capable to remove blood from deep tissue, including: a hollow needle, connected with a barrel to hold blood and maintain low air pressure, and a one-way pump outlet to pump the air out of the barrel. The invention also includes a bloodletting method, includes: placing a hollow needle into the skin wherein the needle is connected with a barrel, and pumping out the air from the needle and the barrel through a one-way pump outlet, thus the blood is pulled out of the tissue with the air pressure difference between the tissue and the barrel space. in order to avoid the puncturing of blood vessels, nerves, and even worse, the pleura of the chest causing pneumothorax, the present invention also proposed an accessory, a guiding needle. The guiding needle is sharp and can puncture through the skin directly. With the guiding needle, the tip of the hollow needle for bloodletting can be blunt but can still be inserted into the skin through the guiding needle hole. When pushing forward within the tissue, the blunt needle tip will not easily pierce the blood vessels, nerves or even pleura. The invention also proposes a method of bloodletting, with the steps including: placing a hollow needle which is connected with a barrel into the skin, exerting a force to push the blood out of the tissue into the hollow needle and barrel. The force can be generated by creating a lower air pressure inside the barrel and the hollow needle, which is in turn done by pumping the air out of the barrel through the one-way pump outlet; or the force can also be generated by cupping on the target skin with a cupping cup; or the force can be generated by inflating an inflatable cuff wrapped around body parts, such as fingers, arms, and legs. The inflatable cuff will compress the tissue and push the blood out.

According to one aspect of the present invention, a bloodletting syringe is provided, including: a hollow needle that can be inserted into the skin; a barrel that is connected to the hollow needle; a pump outlet being connected to the barrel, wherein the pump outlet is a one-way pump outlet, when pumping, the pump outlet is opened, the air is pumped out, when the pumping is stopped, the pump outlet is automatically closed, and the air pressure in the barrel is maintained lower than the pressure outside of the barrel.

Optionally, the hollow needle has different diameters and lengths to accommodate the thicknesses of the skins and tissues.

Optionally, the hollow needle has a sharp tip and is able to puncture through the skin directly.

Optionally, the hollow needle's tip is blunt, and requires a guiding needle with a sharp tip to puncture the skin, and the guiding needle also has a hollow shaft and a handle for users to hold and to prevent the guiding needle from being completely buried into the skin.

Optionally, the hollow needle has multiple holes on the wall.

Optionally, the bloodletting syringe includes a blood-absorbent pad being placed in the barrel.

Optionally, the bloodletting syringe also includes a pumping equipment that is manual or electric driven.

According to another aspect of the present invention, a bloodletting syringe system is provided, including: a hollow needle that can be inserted into the skin; a barrel that is connected to the hollow needle; a mechanical equipment that can be used with the hollow needle and barrel to exert a force to the skins and tissues.

Optionally, the hollow needle has different diameters and lengths to accommodate the thicknesses of the skins and tissues.

Optionally, the hollow needle has a sharp tip and is able to puncture through the skin directly.

Optionally, the hollow needle has a blunt tip and requires a guiding needle with a sharp tip in order to be inserted into the skin, and the guiding needle also has a hollow shaft and a handle for users to hold and to prevent the guiding needle from being completely buried into the skin.

Optionally, the hollow needle has multiple holes on the wall.

Optionally, the bloodletting syringe system includes a pumping equipment that is manual or electric.

Optionally, the mechanical equipment to exert a force to the skins and tissues is a cupping cup, when in use, the cupping cup is placed above or aside of the bloodletting syringe on the skin, followed by pumping the air out of the cup to create a lower air pressure inside of the cup, which pulls the skins and tissues, and the blood in the tissue is squeezed into the hollow needle and the barrel.

Optionally, the mechanical equipment to exert a force to the skins and tissues is an inflatable cuff, when in use, the inflatable cuff is wrapped around a body part beside of the bloodletting syringe being inserted in the body, followed by inflating the cuff to compress the skins and tissues, and squeeze the blood into the hollow needle and barrel.

According to another aspect of the present invention, a bloodletting method by using a bloodletting syringe or blood letting syringe system is provided, including: inserting a hollow needle into the skin wherein the hollow needle is connected to a barrel; and applying a force to squeeze the blood into the hollow needle and barrel; the force is implemented by pumping the air out of the hollow needle and barrel, which creates a pressure difference between the tissue and the space in the hollow needle; or the force is exerted by cupping with a cupping cup on the skin; or the force is exerted by compressing the skins and tissues with an inflatable cuff wrapping around body parts.

Optionally, the method to place the hollow needle through the skin includes puncturing the skin with a guiding needle and inserting the hollow needle into the skin through the guiding needle.

Optionally, the hollow needle has multiple holes on the wall to allow blood to pass through from surrounding tissues into the hollow needle.

Optionally, the barrel includes a blood-absorbent pad to absorb blood.

The bloodletting syringe, bloodletting syringe systems and bloodletting methods of the present invention can be used to remove blood stasis in the muscles and tissues safely and with great control by the medical staff.

### BRIEF DESCRIPTION OF THE DRAWINGS

FIGS. 1A and 1B show the general shape of a lancet and a triangular needle. Their tips are solid, and the lengths are usually only a few millimeters.

FIGS. 2A and 2B show an example of bloodletting cupping and a manual pumping equipment. In FIG. 2A, 210 is a cupping cup and 220 shows a few blood drops that were squeezed out by the cupping suction force.

FIG. 3 shows the parts of a medical syringe that can be used to inject drugs or withdraw blood, including a needle tip 310, a needle shaft 320 with a length about a few centimeters, a needle hub 325, a barrel 330, a plunger seal 340 and a plunger 350.

FIG. 4 shows a structural diagram of a bloodletting syringe, which is based on an embodiment of the present invention, including a one-way pump outlet 410, a barrel 420, a hollow needle 430, and blood-absorbent pad 440 inside the barrel.

FIG. 5A-5B show a hollow needle with a sharp tip and multiple holes on the wall. FIG. 5A is a schematic diagram and FIG. 5B is a real hollow needle picture. 510 is a sharp needle tip, 520 are holes in the needle wall, and 530 is the needle wall.

FIG. 6A-C shows another design of a hollow needle of the bloodletting syringe and a guiding needle used in conjunction with the hollow needle. In FIG. 6A, the tip of the hollow needle 610 is blunt and the wall of the hollow needle has multiple holes 620. The hollow needle is connected with a barrel 420, which is filled with a blood-absorbent pad 440. The barrel is closed with a one-way pump outlet 410, similar in FIG. 4. FIG. 6B shows a guiding tube consisting of a sharp needle tip 640, a hollow shaft 650, and a handle 660 that is designed for users to hold the guiding needle to insert. The handle can also prevent the guiding needle from being buried into the skins completely. FIG. 6C shows how the bloodletting syringe with blunt hollow needle tip and the guiding needle are used together.

FIG. 7 shows an example of the use of a bloodletting syringe system based on the embodiment of the present invention. 710 is a one-way pump outlet on a cupping cup 720. 760 is a hollow needle which is connected with a barrel 750. 740 is a blood-absorbent pad filled in the barrel. 730 is the body parts to get treated with bloodletting.

FIG. 8 is another application example of the bloodletting syringe system. 810 is a one-way pump outlet of the bloodletting syringe, 820 is a cupping cup, 830 is a one-way pump outlet of the cup, 840 is human body part, 850 is a blood-absorbent pad, 860 is a barrel, 870 is a hollow needle, and 880 is a flexible airtight material connecting the bloodletting syringe and the cupping cup.

FIG. 9 is another example of an embodiment of the present patent. In the figure, 910 is the inflatable cuff, 920 is a flexible tube, 930 is a squeezing ball to inflate the cuff, 940 is a valve to release air to deflate the cuff or to seal the air leaks during inflation, 950 is a barrel, 960 is a blood-absorbent pad, 970 is a hollow needle and 980 is the one-way pump outlet.

FIG. 10 is similar to FIG. 9 in design and usage, except that the inflatable cuff has a different shape, that is, there is a hole 1090 on the inflatable cuff for the needle insertion.

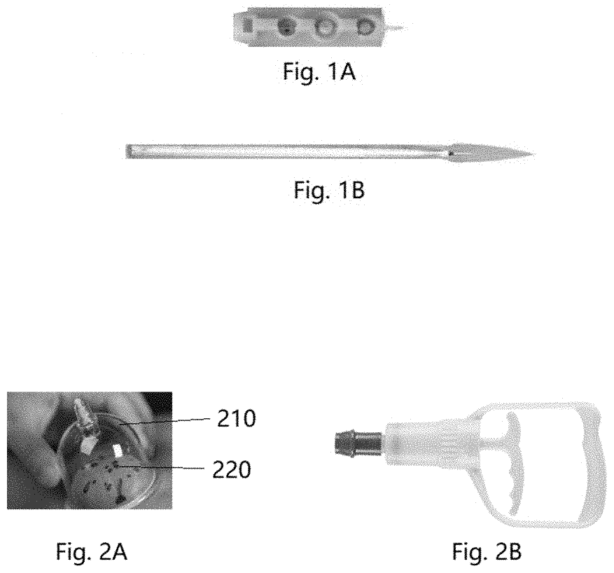

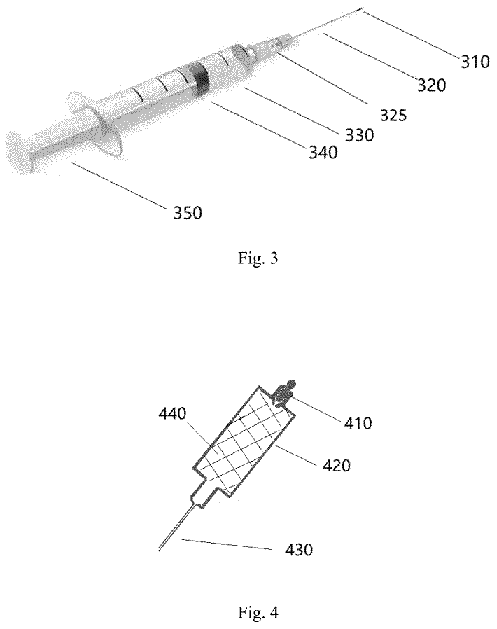

### DETAILED DESCRIPTION

The embodiments of the present invention are described in detail in conjunction with the drawings.

FIG. 4 shows a structural diagram of a bloodletting syringe according to an embodiment of the present invention. It includes a one-way pump outlet 410, a barrel 420, and a hollow needle 430. The operation steps are: the hollow needle 430 is inserted into the skin, connect an external pumping equipment to the one-way pump outlet 410, pump the air out of the barrel 420 and the hollow needle 430 through the one-way pump outlet 410 to create a lower pressure with regard to ambient air pressure. With low air pressure inside the barrel and hollow needle, the blood from the tissue will be squeezed into the barrel. Preferably, the barrel 420 is filled with blood-absorbent pad 440. Pump outlet 410 is a one-way pump outlet. When pumping, the pump outlet is opened, the air is pumped out, when the pumping is stopped, the pump outlet is automatically closed, and the air pressure in the barrel is maintained lower than the pressure outside of the barrel, which creates a force difference between the tissue and the space in the hollow needle and pushes the blood out of the tissue into the needle. The pumping equipment to pump the air out can be manual or automatic.

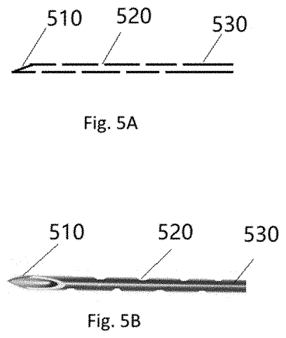

FIG. 5A and FIG. 5B show multiple-hole structure on the wall of a hollow needle (which can be used as a hollow needle 430 in the bloodletting syringe shown in FIG. 4) according to the embodiment of the present invention. FIG. 5A is a schematic graph and FIG. 5B is a picture of a real needle. In the figures, 510 are the tips of the sharp needles, 530 are the needle walls, and 520 are the holes on the needle walls. The porous structure on the needle wall can simultaneously drain blood from multiple locations of the body tissue, increase the efficiency of the bloodletting. However, it should be noted that different tissues have different depth. The hollow needle of the bloodletting syringe will have different lengths and sizes to accommodate the different depth of the tissue to do the bloodletting. Correspondingly, the number of holes in the wall of the hollow needle is affected by the length of the hollow needle and they must be buried under the skin when in use.

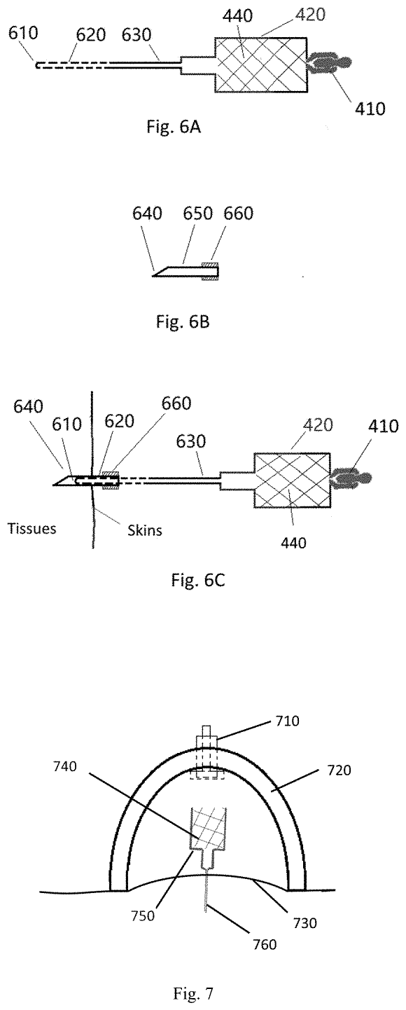

FIG. 6A shows another design of the hollow needle according to the embodiment of the present invention. 630 is a needle shaft, 620 is one of the holes in the wall of the needle shaft. The blood in the tissue can be drained through these holes into the needle and the barrel. The tip of the hollow needle 610 is blunt, thus the insertion of such a needle will not pierce into blood vessels, nerves or chest pleura to cause pneumothorax. However, since the tip of the needle is blunt, it is also not easy to puncture the skin and get into the tissue. Therefore, it requires a guiding needle to be used in conjunction with the blunt-tip needle. FIG. 6B shows such a guiding needle according to an embodiment of the present invention. 640 is a sharp tip of the guiding needle. 650 is a hollow needle shaft. 660 is a handle, convenient for users to hold the guiding tube and insert it into the skin. The handle can also prevent the guiding needle from being buried into the tissue completely. FIG. 6C shows the use of a bloodletting syringe with a guiding needle. The steps are: puncturing the skin with a guiding needle, inserting a hollow needle of a bloodletting syringe through the guiding needle into the tissue, pumping the air out of the barrel to create a lower air pressure inside the barrel and the internal space of the hollow needle than the ambient air pressure.

According to another embodiment of the present invention, a bloodletting syringe system is provided, including: a hollow needle that can be inserted into the skin; a barrel that is connected to the hollow needle, with or without a one-way pump outlet; a mechanical equipment that can be used with the hollow needle and barrel to apply force to the skins and tissues.

FIG. 7 shows an example of the use of a bloodletting syringe system based on the embodiment of the present invention. The steps to use the system include, inserting a hollow needle 760 into the skin, wherein the hollow needle is connected with a barrel 750, placing a cupping cup 720 above the bloodletting syringe onto the skin, pumping the air out of the cupping cup through a one-way pump outlet 710 to create a lower air pressure inside the cup than the atmospheric pressure. The lower air pressure can pull the skin 730 up and squeeze the blood out. In this example, the barrel does not have a pump outlet connected to it. Preferably, the barrel is filled with a blood-absorbent pad 740 to prevent the blood outflow.

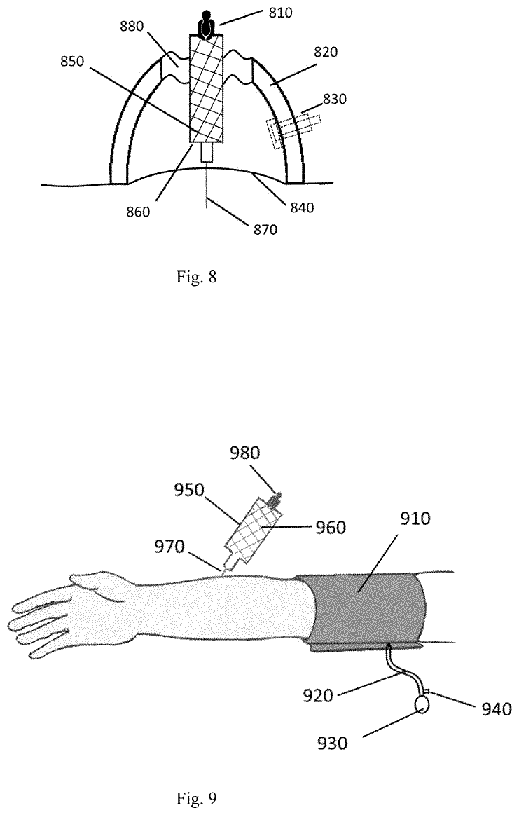

FIG. 8 is another example of the bloodletting syringe system. In this example, the bloodletting syringe and the cupping device are integrated. 810 is a one-way pump outlet of the bloodletting syringe, 820 is a cupping cup, 830 is a one-way pump outlet of the cup, 840 is human skin, 850 is a blood-absorbent pad, 860 is a barrel, 870 is a hollow needle, and 880 is a flexible airtight material connecting the bloodletting syringe and the cupping cup. Since the airtight material 880 is flexible, the bloodletting syringe can move relative to the cupping cup and can be inserted into the skin by external force. When in use, place the bloodletting syringe system onto the skin with the cup covering the skin, insert the hollow needle into the skin, pump the air out of the cupping cup and the barrel through their pump outlets respectively with a pumping equipment, without a specific order. The blood in the human tissue is thus pushed into the needle and the barrel through the suction force generated by the cupping and through the pressure difference between the body tissue and the space inside the bloodletting syringe.

FIG. 9 is another example of an embodiment of the present patent. In the figure, 910 is the inflatable cuff, 920 is a flexible tube, 930 is a squeezing ball to inflate the cuff, 940 is a valve to release air to deflate the cuff or to seal the air leaks during inflation, 950 is a barrel, 960 is a blood-absorbent pad, 970 is a hollow needle and 980 is the one-way pump outlet. When in use, the medical staff need to insert the hollow needle 970 into the skin and wrap the inflatable cuff around a body part near the needle, without a specific order. The inflatable cuff 910 is then inflated by squeezing the squeezing ball and with the valve closed. The inflated cuff compresses the tissue and squeeze the blood out of the tissue into the hollow needle. After the bloodletting is done, pull out the bloodletting syringe, open the valve to release the air inside the cuff and remove the inflatable cuff.

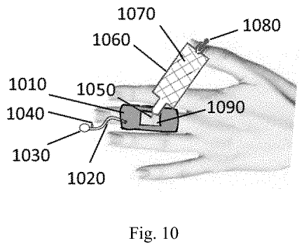

FIG. 10 is similar to FIG. 9 in design and usage, except that the inflatable cuff has a different shape: there is a hole on the inflatable cuff for the needle insertion. The advantage is that the tissue around the needle is squeezed all together for more efficient bloodletting. 1110 is an inflatable cuff with a hole 1090 in it. 1020 is a flexible tube through which to inflate the cuff. 1030 is a squeezing ball. 1040 is a valve to release air when open and to seal the air leaks when closed. 1060 is a barrel that is connected to a hollow needle 1050. 1070 is a blood-absorbent pad inside the barrel. 1080 is a one-way pump outlet.

According to another embodiment of the present invention, a method of bloodletting with the use of bloodletting syringe or bloodletting syringe system is provided, including: inserting a hollow needle into the skin wherein the hollow needle is connected to a barrel, apply force to the skin and tissue to squeeze the blood into the hollow needle and barrel; the force is implemented by pumping the air out of the hollow needle and barrel, which creates a pressure difference between the tissue and the internal space of the bloodletting syringe; or the force is implemented by cupping with a cup on the skin; or the force is implemented by compressing the tissue with an inflatable cuff.

As an example, the method to insert the hollow needle through the skin includes: puncturing the skin with a guiding needle and inserting the hollow needle into the skin through the guiding needle.

As another example, the hollow needle has multiple holes on the needle shaft to allow the blood to pass through.

As another example, the barrel includes a blood-absorbent pad to absorb blood.

The embodiments of the present invention have been described above, and the above description is exemplary, not exhaustive, and is not limited to the disclosed embodiments. Without departing from the scope and spirit of the embodiments described, many of the modifications and changes are obvious to the average technician in the field of technology. Therefore, the scope of protection of the present invention should be based on the scope of protection of claims.

1. A bloodletting syringe, including:
a hollow needle that can be inserted into skins;
a barrel that is connected to the hollow needle;
a pump outlet being connected to the barrel, wherein the pump outlet is a one-way pump outlet, when pumping, the pump outlet is opened, air that is inside of the barrel and the hollow needle is pumped out, when the pumping is stopped, the pump outlet is automatically closed, and air pressure in the barrel is maintained lower than air pressure outside of the barrel.

2. The bloodletting syringe of claim 1, wherein the hollow needle has different diameters and lengths to accommodate different thicknesses of skins and tissues.

3. The bloodletting syringe of claim 1, wherein the hollow needle has a sharp tip and is able to puncture through skins directly.

4. The bloodletting syringe of claim 1, wherein the hollow needle's tip is blunt, and requires a guiding needle with a sharp tip in order to be inserted into skins, and the guiding needle also has a hollow shaft and a handle for users to hold and to prevent the guiding needle from being completely buried into skins.

6. The bloodletting syringe of claim 1, wherein the hollow needle has multiple holes on a wall.

7. The bloodletting syringe of claim 1 also includes:
a blood-absorbent pad being placed in the barrel.

8. The bloodletting syringe of claim 1, further comprising:
a pumping equipment that is manual or electric driven.

9. A bloodletting syringe system, including:
a hollow needle that can be inserted into skins;
a barrel that is connected to the hollow needle, wherein the barrel has or has no pump outlet;
a mechanical equipment that is operable to be used with the hollow needle and barrel to exert force to skins and tissues.

10. The bloodletting syringe system of claim 9, wherein the hollow needle has different diameters and lengths to accommodate different thicknesses of skins and tissues.

11. The bloodletting syringe system of claim 9, wherein the hollow needle has a sharp tip and is able to puncture through skins directly.

12. The bloodletting syringe system of claim 9, wherein the hollow needle has a blunt tip and requires a guiding needle with a sharp tip in order to be inserted into the skin, and the guiding needle also has a hollow shaft and a handle for users to hold and to prevent the guiding needle from being completely buried into the skin.

13. The bloodletting syringe system of claim 9, wherein the hollow needle has multiple holes on a wall.

14. The bloodletting syringe of claim 9, further comprising:
a pumping equipment that is manual or electric driven.

15. The bloodletting syringe of claim 9, wherein the mechanical equipment to exert force to skins and tissues is a cupping cup, when in use, the cupping cup is placed above or aside of the bloodletting syringe on skin, followed by pumping air out to create a lower air press inside of the cup, which lifts and squeezes skins and tissues, and blood is squeezed into the hollow needle and the barrel.

16. The bloodletting syringe of claim 9, wherein the mechanical equipment to exert force to skins and tissues is an inflatable cuff, when in use, the inflatable cuff is wrapped around a body part aside of the bloodletting syringe, followed by inflating the cuff to compress skins and tissues, and squeeze blood into the hollow needle and barrel.

17. A bloodletting method by using a bloodletting syringe during acupuncture treatment, includes:
inserting a hollow needle of a bloodletting syringe into the skin; and
exerting a force to skins and tissues to squeeze blood into the hollow needle and barrel; the force is implemented by pumping air out of the bloodletting syringe which creates a pressure difference between tissues and the space in the bloodletting syringe; or the force is implemented by cupping with a cup on skins; or the force is implemented by compressing skins and tissues with an inflatable cuff.

18. The bloodletting method of claim 17, wherein the method to place the hollow needle through the skin comprising:
puncturing the skin with a guiding needle and inserting the hollow needle into tissues through the guiding needle.

19. The bloodletting method of claim 17, wherein the hollow needle has multiple holes to allow blood to pass through from surrounding tissues.

20. The bloodletting method of claim 17, wherein the barrel comprising:
a blood-absorbent pad being placed in the barrel to absorb blood.
